(19) United States
(12) Patent Application Publication
(10) Pub. No.: US 2025/0205693 A1
(43) Pub. Date: Jun. 26, 2025

(54) CATALYST AND METHOD OF FORMING FORMATE

(71) Applicant: INDUSTRIAL TECHNOLOGY RESEARCH INSTITUTE, Hsinchu (TW)

(72) Inventors: Mao-Lin HSUEH, Tainan City (TW); Yi-Zhen CHEN, Tainan City (TW); Ya Yun LEE, Tainan City (TW)

(73) Assignee: INDUSTRIAL TECHNOLOGY RESEARCH INSTITUTE, Hsinchu (TW)

(21) Appl. No.: 18/987,287

(22) Filed: Dec. 19, 2024

(30) Foreign Application Priority Data
Dec. 20, 2023 (TW) 112149649

(51) Int. Cl. B01J 31/22; B01J 31/18; B01J 31/20; C07C 51/15; C07C 227/16

(52) U.S. Cl. CPC B01J 31/2295; B01J 31/181; B01J 31/20; C07C 51/15; C07C 227/16; B01J 2231/005; B01J 2531/004; B01J 2531/0238; B01J 2531/824

(57) ABSTRACT

A catalyst includes 1 part by mole of a palladium complex and 500 to 10000 parts by mole of an amine compound. The palladium complex has a chemical structure of

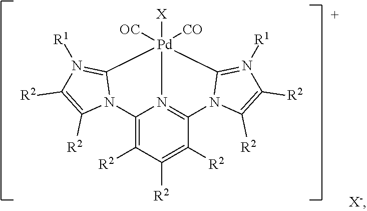

in which X is halogen, each of R¹ is independently C₁₋₈ linear alkyl group or C₃₋₈ cycloalkyl group, and each of R² is independently H or C₁₋₄ alkyl group. The amine compound has a chemical structure of

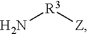

in which R³ is C₁₋₈ alkylene group, and Z is

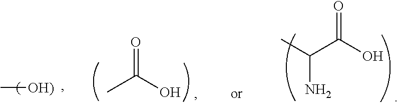

### CROSS REFERENCE TO RELATED APPLICATIONS

The present application is based on, and claims priority from, Taiwan Application Serial Number 112149649, filed on Dec. 20, 2023, the disclosure of which is hereby incorporated by reference herein in its entirety.

### TECHNICAL FIELD

The technical field relates to a catalyst, and a method of forming formate utilizing the catalyst.

### BACKGROUND

Formic acid is one of the most basic organic chemical raw materials, which is widely used in the fields of food preservatives, animal feed additives, textile leathers, dyes, industrial cleaners, rubbers, chemical industries, and the like. In addition, formic acid is considered an excellent compound for use as a hydrogen storage material, and for this reason it has attracted a lot of attention due to the low energy required for the dehydrogenation reaction and its ease of handling.

The conventional method of producing formic acid is the methanol carbonyl synthesis method (also known as the methyl formate method). The methanol and carbon monoxide react in the presence of the catalyst, sodium methoxide, to form methyl formate, which is then hydrolyzed to form formic acid and methanol. The methanol can be recycled and sent into the methyl formate reactor, and the formic acid is finestilled to obtain products with different specifications. However, the conventional method is complex and requires multiple steps.

Accordingly, a novel method is called for, for simplifying the preparation of formic acid.

### SUMMARY

One embodiment of the disclosure provides a catalyst, including 1 part by mole of a palladium complex and 500 to 10000 parts by mole of an amine compound. The palladium complex has a chemical structure of

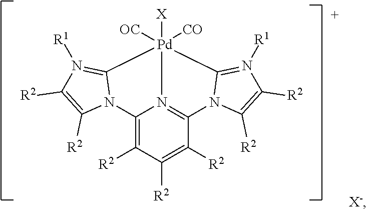

wherein X is halogen, each of R¹ is independently C₁₋₈ linear alkyl group or C₃₋₈ cycloalkyl group, and each of R² is independently H or C₁₋₄ alkyl group. The amine compound has a chemical structure of

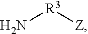

wherein R³ is C₁₋₈ alkylene group, and Z is

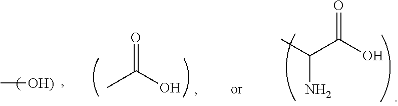

One embodiment of the disclosure provides a method of forming formate, including mixing the described catalyst and a solvent to form a mixture; and introducing carbon dioxide and hydrogen into the mixture to form a formate.

A detailed description is given in the following embodiments.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details.

One embodiment of the disclosure provides a catalyst, including 1 part by mole of a palladium complex and 500 to 10000 parts by mole of an amine compound. If the amount of the amine compound is too little, the reaction efficiency will be too low.

The palladium complex has a chemical structure of

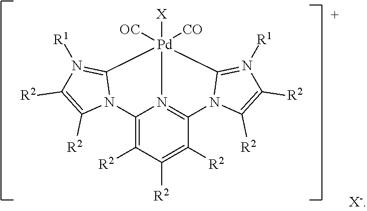

X is halogen, such as chlorine, bromine, or iodine. Each of R¹ is independently C₁₋₈ linear alkyl group or C₃₋₈ cycloalkyl group. For example, R¹ can be butyl group, octyl group, or cyclohexyl group. Each of R² is independently H or C₁₋₄ alkyl group. For example, R² can be H.

The amine compound has a chemical structure of

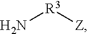

wherein R³ is C₁₋₈ alkylene group, and Z is

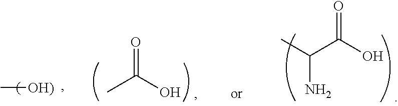

or For example, the amine compound can be lysine, 6-aminohexanoic acid, or ethanolamine.

In some embodiments, the palladium complex has a chemical structure of

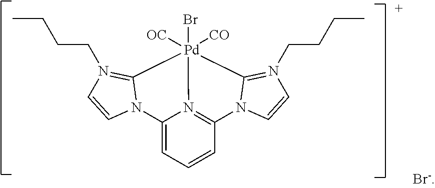

In some embodiments, the palladium complex has a chemical structure of

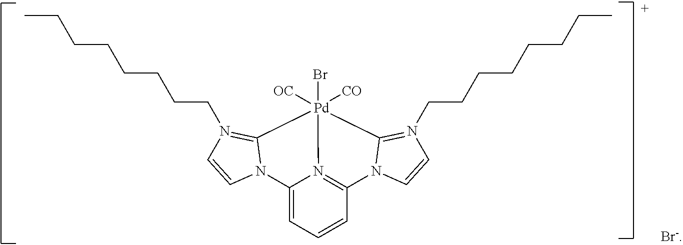

One embodiment of the disclosure provides a method of forming formate, including mixing the described catalyst (containing the palladium complex and the amine compound) and a solvent to form a mixture; and introducing carbon dioxide and hydrogen into the mixture to form a formate. The formate can be further treated to form formic acid and the amine compound, and the amine compound can collocate with the palladium complex again to form the formate.

In some embodiments, carbon dioxide and hydrogen can be sequentially introduced. Alternatively, carbon dioxide and hydrogen can be simultaneously introduced. In general, carbon dioxide and hydrogen may have a pressure ratio of 1:0.5 to 1:5. If the hydrogen amount is too low, the yield of the formate product will be too low. If the hydrogen amount is too high, the yield of the formate product cannot be further increased but hydrogen will be wasted. The reaction temperature can be 100° C. to 180° C., the reaction pressure can be 40 kg/cm² to 100 kg/cm², and the reaction period can be 2 hours to 24 hours. It should be understood that the above reaction condition is only for illustration. One skilled in the art may select suitable reaction conditions as needed that are not limited to the above reaction conditions.

In some embodiments, the solvent includes an organic solvent and water, and the organic solvent includes tetrahydrofuran (THF), 2-methyltetrahydrofuran (MTHF), dimethyl sulfoxide (DMSO), or 1,4-dioxane.

In some embodiments, the formate has a chemical structure of

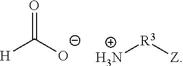

Below, exemplary embodiments will be described in detail so as to be easily realized by a person having ordinary knowledge in the art. The inventive concept may be embodied in various forms without being limited to the exemplary embodiments set forth herein.

### EXAMPLES

### Synthesis Example 1

2,6-dibromopyridine (8.0 g, 33.8 mmol) and 1-butylimidazole (8.39 g, 67.5 mmol) were stirred to react under nitrogen at 150° C. for 20 hours. The reaction result was cooled down to room temperature, and then dissolved in chloroform (70 mL). Ethyl ether was then added to the chloroform solution to form a precipitate. The precipitate was dissolved in methanol and then re-precipitated with ethyl ether, and the dissolving and re-precipitating steps were repeated several times. The precipitate was dried under a reduced pressure to obtain a white solid product [CHNCH-Bu₂]Br₂ (10.7 g, yield=65%). The hydrogen spectrum of the product is shown below: ¹H NMR (CDCl₃): δ 12.00 (s, 2H), 9.25 (s, 2H), 8.77 (d, 2H), 8.30 (t, 1H), 7.46 (s, 2H), 4.60 (t, 4H), 2.01 (quintet, 4H), 1.46 (sextet, 4H), 1.00 (t, 6H).

The ligand [CHNCH-Bu₂]Br₂ (1.46 g, 3.0 mmol) and palladium acetate (Pd(OAc)₂, 0.672 g, 3.0 mmol) were added into anhydrous dimethyl sulfoxide (DMSO, 24 mL), then stirred to react at room temperature for 3 hours, then heated to 50° C. and stirred to react for 12 hours, and then heated to 155° C. and stirred to react for 1 hour. The reaction result was cooled down to room temperature, and poured into dichloromethane (20 mL). The dichloromethane solution was then added into ethyl ether (200 mL) to precipitate a solid. The precipitate was dissolved in chloroform, and ethyl ether was added to the chloroform solution to precipitate a solid. The precipitate was dried under reduced pressure to obtain a yellow-brown solid as a palladium complex product (1.38 g, yield=78%). The hydrogen spectrum of the palladium complex product is shown below: ¹H NMR (CDCl₃): δ 8.66 (s, 2H), 8.46 (d, 2H), 8.33 (t, 1H), 7.23 (s, 2H), 4.60 (t, 4H), 1.78 (quintet, 4H), 1.40 (sextet, 4H), 0.93 (t, 6H). The palladium complex product had a chemical structure of

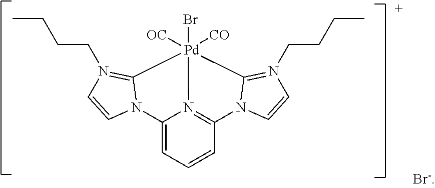

### Synthesis Example 2

2,6-dibromopyridine (8.0 g, 33.8 mmol) and 1-octylimidazole (12.2 g, 67.5 mmol) were stirred to react under nitrogen at 150° C. for 20 hours. The reaction result was cooled down to room temperature, and then dissolved in chloroform (70 mL). Ethyl ether was then added to the chloroform solution to form a precipitate. The precipitate was dissolved in methanol and then re-precipitated with ethyl ether, and the dissolving and re-precipitating steps were repeated several times. The precipitate was dried under a reduced pressure to obtain a yellow solid product [CHNCH-Oc₂]Br₂ (10.1 g, yield=50%). The hydrogen spectrum of the product is shown below: ¹H NMR (CDCl₃): δ 11.88 (s, 2H), 9.23 (s, 2H), 8.71 (d, 2H), 8.29 (t, 1H), 7.47 (s, 2H), 4.56 (t, 4H), 1.99 (quintet, 4H), 1.46 (m, 20H), 0.84 (t, 6H).

The ligand [CHNCH-Oc₂]Br₂ (1.79 g, 3.0 mmol) and palladium acetate (Pd(OAc)₂, 0.672 g, 3.0 mmol) were added into anhydrous DMSO (24 mL), then stirred to react at room temperature for 3 hours, then heated to 50° C. and stirred to react for 12 hours, and then heated to 155° C. and stirred to react for 1 hour. The reaction result was cooled down to room temperature, and poured into dichloromethane (20 mL). The dichloromethane solution was then added into ethyl ether (200 mL) to precipitate a solid. The precipitate was dissolved in chloroform, and ethyl ether was added to the chloroform solution to precipitate a solid. The precipitate was dried under a reduced pressure to obtain a pale yellow solid as a palladium complex product (1.05 g, yield=50%). The hydrogen spectrum of the palladium complex product is shown below: ¹H NMR (DMSO): δ 8.57 (t, 1H), 8.46 (s, 2H), 8.01 (s, 2H), 7.77 (s, 2H), 4.53 (t, 4H), 1.78 (m, 4H), 1.31 (m, 20H), 0.83 (t, 6H). The palladium complex product had a chemical structure of

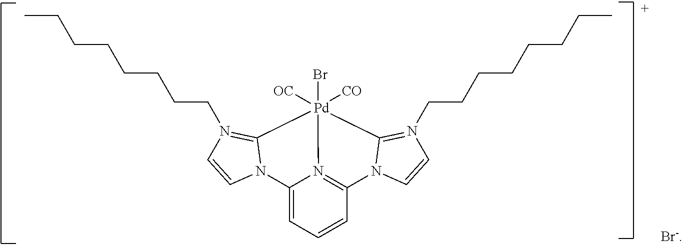

### Comparative Example 1

Lysine (7.31 g, 50 mmol) and palladium acetate Pd(OAc)₂ (4.49 mg, 20 mol) were placed in a reactor. Subsequently, the reactor was evacuated, nitrogen was introduced into the reactor, and the evacuation and introduction steps were repeated three times. Solvent THF/H₂O (1/1, 100 mL, deoxygenated) and internal standard DMF (3.87 mL, 50 mmol, deoxygenated) were added into the reactor, CO₂ (20 kg/cm²) was introduced into the reactor, and the mixture in the reactor was stirred to react at room temperature for 1 hour. Hydrogen was then introduced into the reactor and the pressure in the reactor was built up to 40 kg/cm². The reactor temperature was heated to 140° C., the pressure in the reactor was further built up to 80 kg/cm², and the mixture in the reactor was stirred to react for 24 hours. The reaction result was sampled (about 2 mL) to obtain a two-phase liquid containing a colorless upper layer and a yellow-brown lower layer. De-ionized water (about 1 mL) was added to the two-phase liquid to dissolve the two phases, and the ¹H NMR (D₂O) spectrum of the solution shows that the formate product and the catalyst had a ratio TON (turnover number) of <1. The formate product formed from the lysine had a chemical structure of

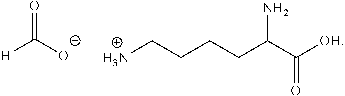

### Comparative Example 2

Lysine (7.31 g, 50 mmol) and palladium chloride PdCl₂ (3.5 mg, 20 mol) were placed in a reactor. Subsequently, the reactor was evacuated, nitrogen was introduced into the reactor, and the evacuation and introduction steps were repeated three times. Solvent THF/H₂O (1/1, 100 mL, deoxygenated) and internal standard DMF (3.87 mL, 50 mmol, deoxygenated) were added into the reactor, CO₂ (20 kg/cm²) was introduced into the reactor, and the mixture in the reactor was stirred to react at room temperature for 1 hour. Hydrogen was then introduced into the reactor and the pressure in the reactor was built up to 40 kg/cm². The reactor temperature was heated to 140° C., the pressure in the reactor was further built up to 80 kg/cm², and the mixture in the reactor was stirred to react for 24 hours. The reaction result was sampled (about 2 mL) to obtain a two-phase liquid containing a colorless upper layer and a yellow-brown lower layer. De-ionized water (about 1 mL) was added to the two-phase liquid to dissolve the two phases, and the ¹H NMR (D₂O) spectrum of the solution shows that the formate product and the catalyst had a ratio TON (turnover number) of <1.

### Comparative Example 3

Lysine (7.31 g, 50 mmol) and palladium trifluoroacetate Pd(TFA)₂ (6.65 mg, 20 mol) were placed in a reactor. Subsequently, the reactor was evacuated, nitrogen was introduced into the reactor, and the evacuation and introduction steps were repeated three times. Solvent THF/H₂O (1/1, 100 mL, deoxygenated) and internal standard DMF (3.87 mL, 50 mmol, deoxygenated) were added into the reactor, CO₂ (20 kg/cm²) was introduced into the reactor, and the mixture in the reactor was stirred to react at room temperature for 1 hour. Hydrogen was then introduced into the reactor and the pressure in the reactor was built up to 40 kg/cm². The reactor temperature was heated to 140° C., the pressure in the reactor was further built up to 80 kg/cm², and the mixture in the reactor was stirred to react for 24 hours. The reaction result was sampled (about 2 mL) to obtain a two-phase liquid containing a colorless upper layer and a yellow-brown lower layer. De-ionized water (about 1 mL) was added to the two-phase liquid to dissolve the two phases, and the ¹H NMR (D₂O) spectrum of the solution shows that the formate product and the catalyst had a ratio TON (turnover number) of 98.

### Example 1

Lysine (7.31 g, 50 mmol) and the palladium complex product in Synthesis Example 1 (11.8 mg, 20 mol) were placed in a reactor. Subsequently, the reactor was evacuated, nitrogen was introduced into the reactor, and the evacuation and introduction steps were repeated three times. Solvent THF/H₂O (1/1, 100 mL, deoxygenated) and internal standard DMF (3.87 mL, 50 mmol, deoxygenated) were added into the reactor, CO₂ (20 kg/cm²) was introduced into the reactor, and the mixture in the reactor was stirred to react at room temperature for 1 hour. Hydrogen was then introduced into the reactor and the pressure in the reactor was built up to 40 kg/cm². The reactor temperature was heated to 140° C., the pressure in the reactor was further built up to 80 kg/cm², and the mixture in the reactor was stirred to react for 24 hours. The reaction result was sampled (about 2 mL) to obtain a two-phase liquid containing a colorless upper layer and a yellow-brown lower layer. De-ionized water (about 1 mL) was added to the two-phase liquid to dissolve the two phases, and the ¹H NMR (D₂O) spectrum of the solution shows that the formate product and the catalyst had a ratio TON of 1650.

The reaction result such as the two-phase liquid containing the colorless upper layer (having THF and the palladium complex) and the lower layer (having water and the formate product) were separated. The colorless upper layer (having THF and the palladium complex) was placed into the reactor. Water (50 mL, deoxygenated) and lysine (7.31 g) were added into the reactor, CO₂ (20 kg/cm²) was introduced into the reactor, and the mixture in the reactor was stirred to react at room temperature for 1 hour. Hydrogen was then introduced into the reactor and the pressure in the reactor was built up to 40 kg/cm². The reactor temperature was heated to 140° C., the pressure in the reactor was further built up to 80 kg/cm², and the mixture in the reactor was stirred to react for 24 hours. The reaction result was sampled (about 2 mL) to obtain a two-phase liquid containing a colorless upper layer and a yellow-brown lower layer. De-ionized water (about 1 mL) was added to the two-phase liquid to dissolve the two phases, and the ¹H NMR (D₂O) spectrum of the solution shows that the formate product and the catalyst had a ratio TON of 1610. As known from above, the palladium complex of the disclosure could be repeatedly used.

### Comparative Example 4

Lysine (7.31 g, 50 mmol) and a ruthenium complex (11.7 mg, 20 mol) were placed in a reactor. Subsequently, the reactor was evacuated, nitrogen was introduced into the reactor, and the evacuation and introduction steps were repeated three times. Solvent THF/H₂O (1/1, 100 mL, deoxygenated) and internal standard DMF (3.87 mL, 50 mmol, deoxygenated) were added into the reactor, CO₂ (20 kg/cm²) was introduced into the reactor, and the mixture in the reactor was stirred to react at room temperature for 1 hour. Hydrogen was then introduced into the reactor and the pressure in the reactor was built up to 40 kg/cm². The reactor temperature was heated to 140° C., the pressure in the reactor was further built up to 80 kg/cm², and the mixture in the reactor was stirred to react for 24 hours. The reaction result was sampled (about 2 mL) to obtain a two-phase liquid containing a colorless upper layer and a yellow-brown lower layer. De-ionized water (about 1 mL) was added to the two-phase liquid to dissolve the two phases, and the ¹H NMR (D₂O) spectrum of the solution shows that the formate product and the catalyst had a ratio TON of 2380. The ruthenium complex was Ru-MACHOR-BH, which had a chemical structure of

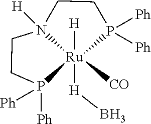

and was commercially available from Aldrich.

The reaction result such as the two-phase liquid containing the colorless upper layer (having THF and the ruthenium complex) and the lower layer (having water and the formate product) were separated. The colorless upper layer (having THF and the ruthenium complex) was placed into the reactor. Water (50 mL, deoxygenated) and lysine (7.31 g) were added into the reactor, CO₂ (20 kg/cm²) was introduced into the reactor, and the mixture in the reactor was stirred to react at room temperature for 1 hour. Hydrogen was then introduced into the reactor and the pressure in the reactor was built up to 40 kg/cm². The reactor temperature was heated to 140° C., the pressure in the reactor was further built up to 80 kg/cm², and the mixture in the reactor was stirred to react for 24 hours. The reaction result was sampled (about 2 mL) to obtain a two-phase liquid containing a colorless upper layer and a yellow-brown lower layer. De-ionized water (about 1 mL) was added to the two-phase liquid to dissolve the two phases, and the ¹H NMR (D₂O) spectrum of the solution shows that the formate product and the catalyst had a ratio TON of 560. As known from above, the activity of the reused ruthenium complex was greatly reduced.

### Example 2

Lysine (7.31 g, 50 mmol) and the palladium complex product in Synthesis Example 2 (14.0 mg, 20 mol) were placed in a reactor. Subsequently, the reactor was evacuated, nitrogen was introduced into the reactor, and the evacuation and introduction steps were repeated three times. Solvent THF/H₂O (1/1, 100 mL, deoxygenated) and internal standard DMF (3.87 mL, 50 mmol, deoxygenated) were added into the reactor, CO₂ (20 kg/cm²) was introduced into the reactor, and the mixture in the reactor was stirred to react at room temperature for 1 hour. Hydrogen was then introduced into the reactor and the pressure in the reactor was built up to 40 kg/cm². The reactor temperature was heated to 140° C., the pressure in the reactor was further built up to 80 kg/cm², and the mixture in the reactor was stirred to react for 24 hours. The reaction result was sampled (about 2 mL) to obtain a two-phase liquid containing a colorless upper layer and a yellow-brown lower layer. De-ionized water (about 1 mL) was added to the two-phase liquid to dissolve the two phases, and the ¹H NMR (D₂O) spectrum of the solution shows that the formate product and the catalyst had a ratio TON of 1575.

### Example 3

Lysine (7.31 g, 50 mmol) and the palladium complex product in Synthesis Example 1 (176.9 mg, 300 mol) were placed in a reactor. Subsequently, the reactor was evacuated, nitrogen was introduced into the reactor, and the evacuation and introduction steps were repeated three times. Solvent THF/H₂O (1/1, 100 mL, deoxygenated) and internal standard DMF (3.87 mL, 50 mmol, deoxygenated) were added into the reactor, CO₂ (20 kg/cm²) was introduced into the reactor, and the mixture in the reactor was stirred to react at room temperature for 1 hour. Hydrogen was then introduced into the reactor and the pressure in the reactor was built up to 40 kg/cm². The reactor temperature was heated to 140° C., the pressure in the reactor was further built up to 80 kg/cm², and the mixture in the reactor was stirred to react for 24 hours. The reaction result was sampled (about 2 mL) to obtain a two-phase liquid containing a colorless upper layer and a yellow-brown lower layer. De-ionized water (about 1 mL) was added to the two-phase liquid to dissolve the two phases, and the ¹H NMR (D₂O) spectrum of the solution shows that the formate product and the catalyst had a ratio TON of 2900.

### Example 4

Lysine (7.31 g, 50 mmol) and the palladium complex product in Synthesis Example 1 (11.8 mg, 20 mol) were placed in a reactor. Subsequently, the reactor was evacuated, nitrogen was introduced into the reactor, and the evacuation and introduction steps were repeated three times. Solvent MTHF/H₂O (1/1, 100 mL, deoxygenated) was added into the reactor, CO₂ (20 kg/cm²) was introduced into the reactor, and the mixture in the reactor was stirred to react at room temperature for 1 hour. Hydrogen was then introduced into the reactor and the pressure in the reactor was built up to 40 kg/cm². The reactor temperature was heated to 140° C., the pressure in the reactor was further built up to 80 kg/cm², and the mixture in the reactor was stirred to react for 24 hours. The reaction result was sampled (about 4 mL) to obtain a two-phase liquid containing a colorless upper layer and a yellow-brown lower layer. Internal standard DMF (77 L, 1 mmol) was added to the lower layer (about 1 mL), and the ¹H NMR (D₂O) spectrum of the lower layer shows that the formate product and the catalyst had a ratio TON of 1550.

### Example 5

Lysine (7.31 g, 50 mmol) and the palladium complex product in Synthesis Example 1 (11.8 mg, 20 mol) were placed in a reactor. Subsequently, the reactor was evacuated, nitrogen was introduced into the reactor, and the evacuation and introduction steps were repeated three times. Solvent DMSO/H₂O (1/1, 100 mL, deoxygenated) and internal standard DMF (3.87 mL, 50 mmol, deoxygenated) were added into the reactor, CO₂ (20 kg/cm²) was introduced into the reactor, and the mixture in the reactor was stirred to react at room temperature for 1 hour. Hydrogen was then introduced into the reactor and the pressure in the reactor was built up to 40 kg/cm². The reactor temperature was heated to 140° C., the pressure in the reactor was further built up to 80 kg/cm², and the mixture in the reactor was stirred to react for 24 hours. The reaction result was sampled (about 2 mL) to obtain a two-phase liquid containing a pale-yellow upper layer and a yellow-brown lower layer. De-ionized water (about 1 mL) was added to the two-phase liquid to dissolve the two phases, and the ¹H NMR (D₂O) spectrum of the solution shows that the formate product and the catalyst had a ratio TON of 1750.

### Example 6

Lysine (7.31 g, 50 mmol) and the palladium complex product in Synthesis Example 1 (11.8 mg, 20 mol) were placed in a reactor. Subsequently, the reactor was evacuated, nitrogen was introduced into the reactor, and the evacuation and introduction steps were repeated three times. Solvent 1,4-dioxane/H₂O (1/1, 100 mL, deoxygenated) and internal standard DMF (3.87 mL, 50 mmol, deoxygenated) were added into the reactor, CO₂ (20 kg/cm²) was introduced into the reactor, and the mixture in the reactor was stirred to react at room temperature for 1 hour. Hydrogen was then introduced into the reactor and the pressure in the reactor was built up to 40 kg/cm². The reactor temperature was heated to 140° C., the pressure in the reactor was further built up to 80 kg/cm², and the mixture in the reactor was stirred to react for 24 hours. The reaction result was sampled (about 2 mL) to obtain a two-phase liquid containing a pale-yellow upper layer and a yellow-brown lower layer. De-ionized water (about 1 mL) was added to the two-phase liquid to dissolve the two phases, and the ¹H NMR (D₂O) spectrum of the solution shows that the formate product and the catalyst had a ratio TON of 1025.

### Example 7

6-aminohexanoic acid (AHA, 6.56 g, 50 mmol) and the palladium complex product in Synthesis Example 1 (11.8 mg, 20 mol) were placed in a reactor. Subsequently, the reactor was evacuated, nitrogen was introduced into the reactor, and the evacuation and introduction steps were repeated three times. Solvent THF/H₂O (1/1, 100 mL, deoxygenated) and internal standard DMF (3.87 mL, 50 mmol, deoxygenated) were added into the reactor, CO₂ (20 kg/cm²) was introduced into the reactor, and the mixture in the reactor was stirred to react at room temperature for 1 hour. Hydrogen was then introduced into the reactor and the pressure in the reactor was built up to 40 kg/cm². The reactor temperature was heated to 140° C., the pressure in the reactor was further built up to 80 kg/cm², and the mixture in the reactor was stirred to react for 24 hours. The reaction result was sampled (about 2 mL) to obtain a two-phase liquid containing a colorless upper layer and a colorless lower layer. De-ionized water (about 1 mL) was added to the two-phase liquid to dissolve the two phases, and the ¹H NMR (D₂O) spectrum of the solution shows that the formate product and the catalyst had a ratio TON of 1175. The formate formed from AHA had a chemical structure of

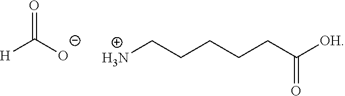

### Example 8

Ethanolamine (ETA, 3.05 g, 50 mmol) and the palladium complex product in Synthesis Example 1 (11.8 mg, 20 mol) were placed in a reactor. Subsequently, the reactor was evacuated, nitrogen was introduced into the reactor, and the evacuation and introduction steps were repeated three times. Solvent THF/H₂O (1/1, 100 mL, deoxygenated) and internal standard DMF (3.87 mL, 50 mmol, deoxygenated) were added into the reactor, CO₂ (20 kg/cm²) was introduced into the reactor, and the mixture in the reactor was stirred to react at room temperature for 1 hour. Hydrogen was then introduced into the reactor and the pressure in the reactor was built up to 40 kg/cm². The reactor temperature was heated to 140° C., the pressure in the reactor was further built up to 80 kg/cm², and the mixture in the reactor was stirred to react for 24 hours. The reaction result was sampled (about 2 mL) to obtain a two-phase liquid containing a colorless upper layer and a colorless lower layer. De-ionized water (about 1 mL) was added to the two-phase liquid to dissolve the two phases, and the ¹H NMR (D₂O) spectrum of the solution shows that the formate product and the catalyst had a ratio TON of 2025. The formate formed from ETA had a chemical structure of

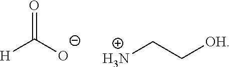

### Example 9

ETA (3.05 g, 50 mmol) and the palladium complex product in Synthesis Example 1 (11.8 mg, 20 mol) were placed in a reactor. Subsequently, the reactor was evacuated, nitrogen was introduced into the reactor, and the evacuation and introduction steps were repeated three times. Solvent MTHF/H₂O (1/1, 100 mL, deoxygenated) was added into the reactor, CO₂ (20 kg/cm²) was introduced into the reactor, and the mixture in the reactor was stirred to react at room temperature for 1 hour. Hydrogen was then introduced into the reactor and the pressure in the reactor was built up to 40 kg/cm². The reactor temperature was heated to 140° C., the pressure in the reactor was further built up to 80 kg/cm², and the mixture in the reactor was stirred to react for 24 hours. The reaction result was sampled (about 4 mL) to obtain a two-phase liquid containing a colorless upper layer and a yellow-brown lower layer. Internal standard DMF (77 μL, 1 mmol) was added to the lower layer (about 1 mL), and the ¹H NMR (D₂O) spectrum of the lower layer shows that the formate product and the catalyst had a ratio TON of 1850.

It will be apparent to those skilled in the art that various modifications and variations can be made to the disclosed methods and materials. It is intended that the specification and examples be considered as exemplary only, with the true scope of the disclosure being indicated by the following claims and their equivalents.

What is claimed is:

1. A catalyst, comprising:
1 part by mole of a palladium complex; and
500 to 10000 parts by mole of an amine compound,
wherein the palladium complex has a chemical structure of 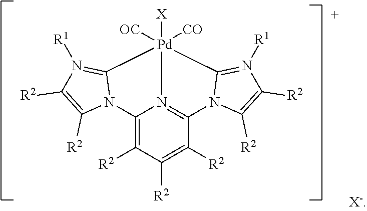
wherein X is halogen,
each of R¹ is independently C₁₋₈ linear alkyl group or C₃₋₈ cycloalkyl group, and
each of R² is independently H or C₁₋₄ alkyl group; and
wherein the amine compound has a chemical structure of 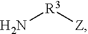
wherein R³ is C₁₋₈ alkylene group, and
Z is

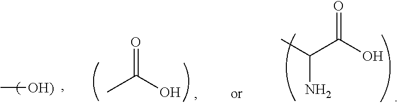

2. The catalyst as claimed in claim 1, wherein the palladium complex has a chemical structure of

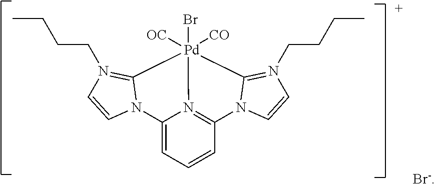

3. The catalyst as claimed in claim 1, wherein the palladium complex has a chemical structure of

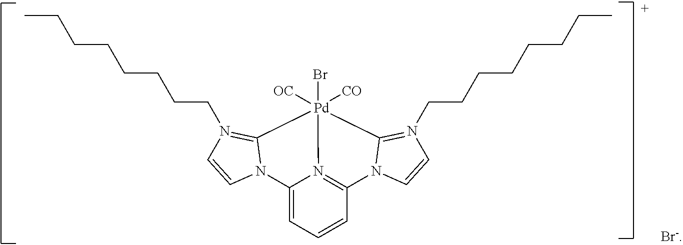

4. The catalyst as claimed in claim 1, wherein the amine compound comprises lysine, 6-aminohexanoic acid, or ethanolamine.

5. A method of forming formate, comprising:
mixing the catalyst as claimed in claim 1 and a solvent to form a mixture; and
introducing carbon dioxide and hydrogen into the mixture to form a formate.

6. The method as claimed in claim 5, wherein the solvent comprises an organic solvent and water, and the organic solvent comprises tetrahydrofuran, 2-methyltetrahydrofuran, dimethyl sulfoxide, or 1,4-dioxane.

7. The method as claimed in claim 5, wherein the formate has a chemical structure of [chemical structure]
